(19) United States
(12) Patent Application Publication
(10) Pub. No.: US 2017/0209681 A1
(43) Pub. Date: Jul. 27, 2017

(54) Body art transfer device

(71) Applicants: Sven Dobler, Huntington, NY (US); Blaine Stambaugh, Signal Mountain, TN (US); Herve Ferrec, Epinay-sur-seine (FR)

(72) Inventors: Sven Dobler, Huntington, NY (US); Blaine Stambaugh, Signal Mountain, TN (US); Herve Ferrec, Epinay-sur-seine (FR)

(21) Appl. No.: 15/530,962

(22) Filed: Mar. 29, 2017

Related U.S. Application Data
(63) Continuation of application No. 13/999,026, filed on Jan. 3, 2014.
Application No. 13/999,026 is a continuation of application No. 13/135,038, filed on Jun. 23, 2011, now abandoned.
Application No. 13/135,038 is a continuation of application No. 12/384,447, filed on Apr. 4, 2009, now abandoned.
(60) Provisional application No. 61/399,153, filed on Jul. 8, 2010.
(60) Provisional application No. 61/072,974, filed on Apr. 4, 2008.

(51) Int. Cl. A61M 37/00; A61K 8/92; A61K 8/25; A61K 8/67; A61K 8/37; A61K 8/34; A61Q 1/02; A61K 8/81

(52) U.S. Cl. CPC A61M 37/0076; A61M 37/00; A61Q 1/02; A61Q 1/025; A61K 8/922; A61K 8/92; A61K 8/8182; A61K 8/678; A61K 8/37; A61K 8/347; A61K 8/342; A61K 8/25

(57) ABSTRACT

A body art transfer device is disclosed which comprises a base support having a generally planar shape, a substantially nonabsorbent textured surface, and a surface area, a body art coating upon the nonabsorbent textured surface, the coating having a pigmented composition of oils and waxes having a thickness of uniform homogeneous consistency and an amorphous non-crystalline structure, and a cover for covering the body art coating and for forming stilting of the body art coating between the nonabsorbent textured surface of the base support and the cover, the stilting for preventing wicking of body art coating, and the body art coating for releasing from the base support when the cover is removed and the body art covering is placed against skin of a user.

### CROSS REFERENCE TO RELATED APPLICATION

This continuation patent application claims priority as a continuation of patent application having Ser. No. 13/999,026, filed Jan. 3, 2014, which claims priority as a continuation to the non-provisional patent application having Ser. No. 13/135,038, filed Jun. 23, 2011, which claims priority to the provisional patent application having Ser. No. 61/399,153, filed Jul. 8, 2010, and this application claims priority as a continuation to the non-provisional patent application having Ser. No. 12/384,447, filed Apr. 4, 2009, which claims priority to the provisional patent application having Ser. No. 61/072,974, filed Apr. 4, 2008, all of these applications are owned by a common assignee.

### FIELD OF THE INVENTION

Tattoos and other forms of body art have grown increasingly popular in today's culture both in North America and Europe. Body art allows persons to express themselves to the public upon visible portions of the person's body and more privately on portions of the person's body not usually seen. Body art can have various colors and designs of all descriptions. Body art can be upon a digit, one limb, the torso, the face, or a combination of them.

As a subset of that enthusiasm for body art, temporary tattoos have also seen gains in popularity and usage among people of all ages. Temporary tattoos allow a person to decorate their body with art that does not remain indefinitely as would a normal tattoo. A temporary tattoo adheres to the skin surface somewhat like an ink pattern or like a sheet of a printed design. Some temporary tattoos use henna and other substances as inks. Similar to normal tattoos, temporary tattoos come in numerous colors and include all kinds of designs. Normal tattoos provide art upon a person's body permanently. Such tattoos can only be removed using surgical procedures. The temporary tattoos also provide art upon a body; temporary tattoos generally fade in time with inks and fall off the skin in time with adhered sheets. Temporary tattoos as a design from a sheet initially provide details in the design. Over time though, temporary tattoos lose their design details as a design sheet crumbles as the underlying skin flexes.

### BACKGROUND OF THE INVENTION

This body art transfer device relates to the manufacture of a human skin decoration sheet and more specifically to a device that transfers a non-crystalline composition, that is, semi-solid, to the skin of a user in a measured amount by a single application.

### SUMMARY OF THE INVENTION

A body art transfer device, in accordance with the present disclosure, permits application of a premeasured amount of body art composition, preferably in a single application, with relative ease, while possessing an acceptable wear characteristic. The body art transfer device of the present disclosure broadly comprises a base support having a nonabsorbent surface, and a coating of an easily transferable body art composition coating having an amorphous, non-crystalline form and a thickness in a range of between 0.5 mils and 5 mils. The embossed area which contains the body art composition is then overlaid with a protective cover. A unique aspect of the device allows a consumer to select and to apply body art temporarily upon their skin with easy removal later.

Various cosmetic printing processes apply the body art to selected substrates for eventual usage by consumers. The printing processes provide the design and color of the art while the substrates retain the image of the tattoo until usage.

The device also retains, protects, and transfers a detailed design from a planar substrate onto a person's skin. The device can be deployed or used as fashion accessories, cosmetic designs, sports team logos and mascots, brand logos, cultural symbols, icons both religious and non-religious, names, advertising specialties, toys, and the like.

Numerous features and advantages of the present disclosure will be readily apparent to those of ordinary skill in the art upon a reading of the following detailed description of the presently preferred, but nonetheless illustrative, embodiment of the present disclosure when taken in conjunction with the accompanying drawings. Before explaining the current embodiment of the disclosure in detail, it is to be understood that the disclosure is not limited in its application to the details of construction and to the arrangements of the components set forth in the following description or illustrated in the drawings. The device is capable of other embodiments and of being practiced and carried out in various ways. Also, the phraseology and terminology employed herein are for the purpose of description and should not be regarded as limiting.

Therefore the object of the present disclosure is to provide a body art transfer device that retains, protects, and transfers a detailed printed design from a planar substrate onto a person's skin.

Another object of the body art transfer device is to provide a fragrance with a printed design applied to a person's skin.

Another object of the body art transfer device is to provide a printed design that temporarily remains upon a person's skin.

Another object of the body art transfer device is to provide a printed design that removes readily from a person's skin.

Another object of the body art transfer device is to provide a printed design that avoids damaging a person's skin.

Another object of the body art transfer device is to provide a printed design in a broad range of colors.

Another object of the body art transfer device is to provide a printed design that includes shimmer, glitter, and fluorescent pigments.

Another object of the body art transfer device is for the device to use various pigments, minerals, or silicone in a dry form.

Another object of the body art transfer device is to provide multiple layers that avoid the adverse effects of offset.

Another object of the body art transfer device is to use at least one layer with an embossed pattern.

Another object of the body art transfer device is to seal the perimeter of its layers against leakage yet allow for easy opening by a user.

Another object of the body art transfer device is to use either woven or non-woven materials in its construction.

And lastly, another object of the body art transfer device is to provide a design upon a clear substrate that allows a user a visual aid to apply the design to the skin of the user.

These together with other objects of the disclosure, along with the various features of novelty that characterize the disclosure, are pointed out with particularity in the claims annexed to and forming a part of this disclosure. For a better understanding of the disclosure, its operating advantages and the specific objects attained by its uses, reference should be had to the accompanying drawings and descriptive matter in which there are illustrated preferred embodiments.

### BRIEF DESCRIPTION OF THE DRAWINGS

In referring to the drawings,

FIG. 1 shows a perspective view of the present disclosure being used;

FIG. 2 shows a top view of the present disclosure with two forms of indicia;

FIG. 3 provides a perspective view of the top layer of the present disclosure;

FIG. 4 shows a sampler embodiment of the disclosure in plan view;

FIG. 5 illustrates a detailed depiction of a tight grid, or cross hatch, texture pattern with an application of liquid fragrance material;

FIG. 6 illustrates a detailed magnified depiction of a quad cell-type texture pattern with an application of liquid fragrance material;

FIG. 7 illustrates a detailed depiction of a wide grid, or dot matrix-type, texture pattern with an application of liquid fragrance material;

FIG. 8 illustrates a detailed depiction of a random dot pattern applied to the base coating layer through the use of an atomizer and an application of liquid fragrance material; and,

FIG. 9 shows in a detailed view the interaction of liquid fragrance materials with adjoining surface texture.

The same reference numerals refer to the same parts throughout the various figures.

### DESCRIPTION OF THE PREFERRED EMBODIMENT

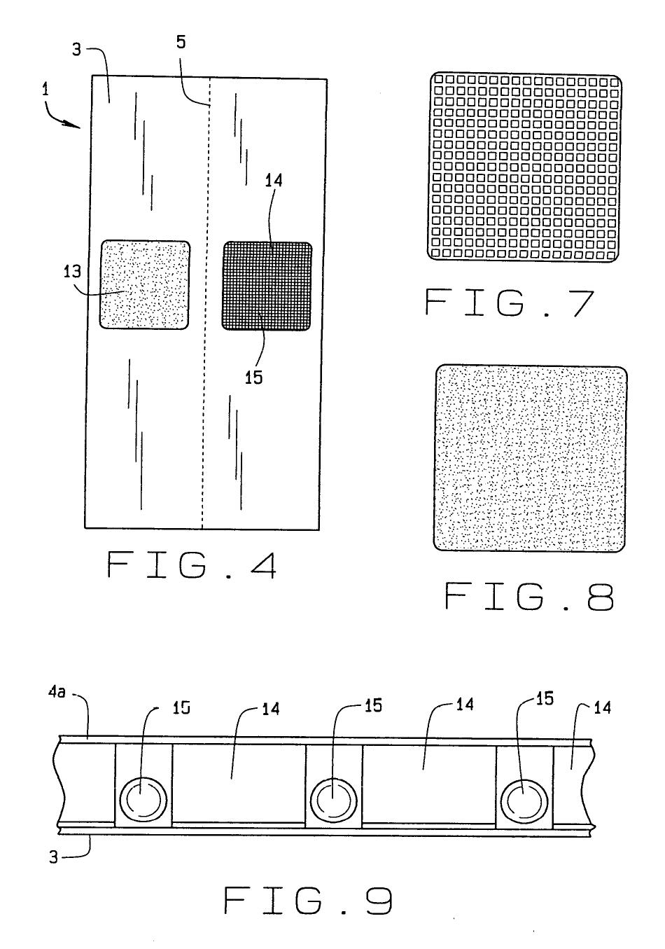

The present art overcomes the prior art limitations by assembling a device that readily delivers the art of a tattoo from a printed substrate onto the skin of a consumer. FIG. 1 shows a consumer P grasping the device 1 of the present disclosure and placing it upon his cheek. The device has art, as at 2, printed upon a transparent textured material thus allowing the art to be seen in this figure.

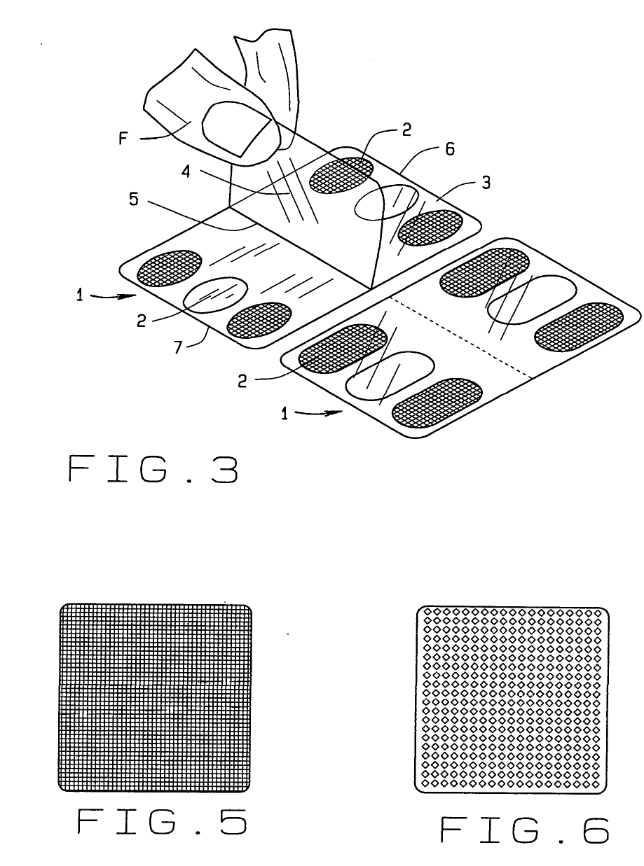

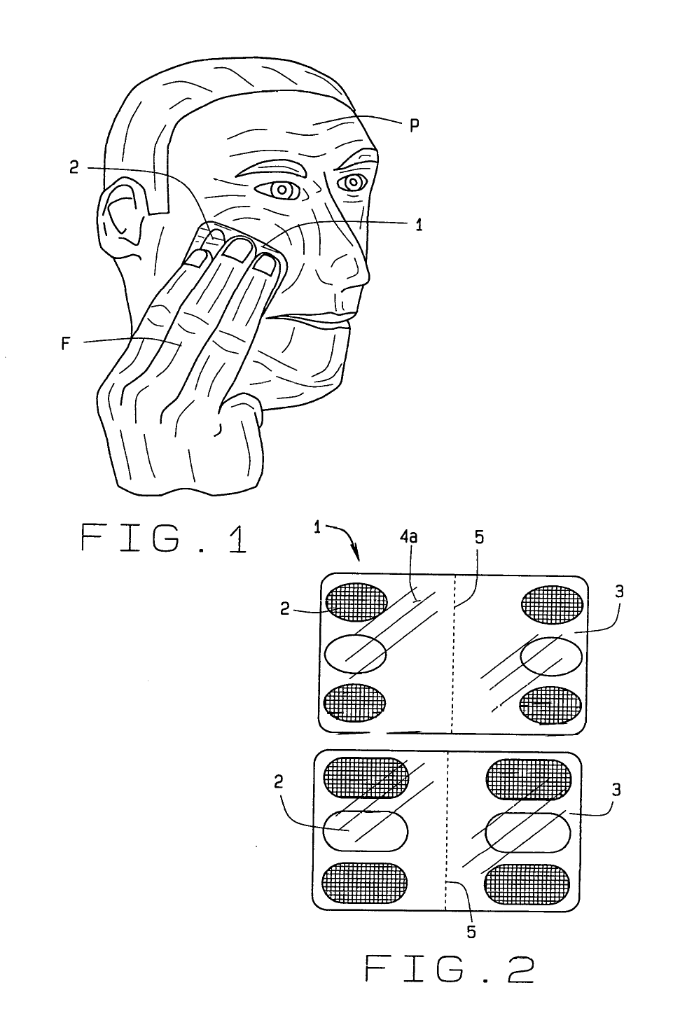

FIG. 2 shows a top view of the device with two versions of art 2. The construction of the device remains similar though the art can vary. The device has a base support 3 substantially rectangular in shape and planar in form. The base support 3 has a substantially nonabsorbent textured surface and a surface area. The nonabsorbent textured surface does not wick oils into the material of the base support 3 but rather repels them. The nonabsorbent textured surface does not have a pattern but rather has an irregular arrangement of disruptions to the plane of the surface. The textured surface includes a plurality of projections forming the irregular arrangement.

Here the base support 3 has rounded corners and sufficient height for three rows of art. The art 2 rests upon the base support 3 and may have various colors, shapes, designs, and appearances generally suitable for use as tattoos and for display upon a person's body. In the preferred embodiment, the base support 3 is generally opaque. In an alternate embodiment, the base support 3 has transparent construction which aids the user to envision the placement of the art 2 as a tattoo and to see its effect before temporarily adhering it the person's skin. The art 2 is separated about a fold line as at 5, generally centered upon the back. Upon the art 2 and extending across the entire base support 3, the device 1 has a cover 4, generally transparent though a reflection of the cover appears as at 4a.

The instances of art 2, that is tattoos, utilize a skin friendly formula of ingredients. This formulation of ingredients shares similarities to color cosmetics such as costume makeup but has heavier concentrations of pigment and a higher oil to wax ratio. The formulation temporarily adheres to skin and clearly displays coloration and designs upon a skin colored background. The formulation also includes alternate pigments and ingredients for shimmer, glitter, fluorescence, mirror, and DAY-GLO attributes. In an alternate formulation, pigments, minerals, and silicone combine in select proportions for usage as a more dry application upon a person's skin. In a further alternate formulation, fragrance augments the pigments, oils, and waxes of previous formulations and the fragrance includes essential oils, aqueous fragrances, and the like.

To maintain or preserve the amorphous structure, the body art composition cannot be heated above its melting temperature, and then allowed to cool and solidify. This method inherently causes the composition to crystallize. Accordingly, any method may be used to form a body art coating on a base surface having the desired thickness, so long as the composition is not liquid at the outset and is not heated above its melting temperature. If the composition is crystalline, it must first be converted to a non-crystalline, amorphous state. This conversion preferably occurs by the application of mechanical energy using, for example, blending or stirring equipment at controlled mixing speeds, to avoid heating the composition above its melting temperature. The body art composition should be mixed relatively slowly to maintain a highly viscous state until a uniform, homogeneous consistency is reached, without melting the composition. Any commercially available mixer may be used for this purpose. The mixing operation modifies the crystalline structure into an amorphous structure.

The amorphous body art composition is then applied to the base support without melting it, generally at a thickness of between 0.5 mils to 5.0 mils. It may be applied by hand-coating, brushing, or painting, or by using any commercially available coating device. It is preferable to apply the body art coating in an unadulterated state, more preferably in a thickness of between 1 mil to 3 mils. The projections of the textured surface have sufficient height about the surface so that the projections extend through the coating applied to the base support. In the preferred embodiment, the projections occupy at least three percent of the base support's surface area. A preferred coating method is screen printing, with the screen printing operation conducted by hand or by using a manual or power-operated commercial press having either a flat screen or a cylindrical screen. The screen may be made of silk, plastic, or metallic mesh. The screen may have an open mesh, with a mesh count of preferably between 80 to 420 per lineal inch. The screen printing is performed in the absence of heat. The body art composition, in coating form, as described above, transfers a tattoo or other art readily from the base support to the skin of a user.

The device 1 has its construction further shown in FIG. 3 where the fingers F of a person P grasp the cover 4. The fingers F typically grasp a rounded corner of the cover 4 and detach it from the base support 3. The cover 4 lifts from the base support 3 from one lateral edge as at 6 towards the opposite lateral edge as at 7. Here the base support 3 has separated from a lateral edge 6 and the cover 4 lifts off the base support to approximately the fold line 5.

Partial lifting of the cover 4 reveals one embodiment of the construction of the device 1. The art 2 has its coloration and design provided by a formulation of ingredients as described above. Prior to its application, the art 2 is placed upon the base support 3, covered, stored, shipped, displayed, and then sold to a person. During those preceding steps, the art 2 remains vulnerable to disturbance, shifting, and offset of ingredients. Offset of ingredients occurs when various ingredients spread or wick into adjacent layers thus diluting the art 2. To retain the art 2 as designed and to prevent offset, the preferred embodiment includes at least two planar layers that have barrier properties which inhibit oils and waxes in the formulation from wicking into a substrate or layer. At least one of the layers has an embossed pattern formed thereon. The embossments rise from about 0.002 inch to about 0.006 inch above the surface of a layer and extend over at least 3% through about 5% to about 75% of the surface of a layer.

In an alternate embodiment, at least one of the layers has a texture from its inherent material properties. In another alternate embodiment, at least one of the layers has a coating printed or deposited thereon that creates a texture for the layer. The coating remains essentially inert and non-reactive with the formulation. The embossments, inherent texture, and coated texture increase the retention of the formulation upon the layers before transfer of the art 2 to skin, induce transfer effects, and ease the deposit of the formulation upon the skin of a person.

Generally, the art 2 is printed upon at least one of the layers on either the textured or embossed portion or upon the smooth or non-treated portion. The smooth portion generally opposes the textured or embossed portion when two layers have adjacent positions. The art 2, as tattoos, forms upon at least one layer using kiss cut or through cut methods of printing. Following printing of the art 2 upon at least one layer, the layers undergo assembly into an aligned stack and then sealing by heat or glue upon at least a portion of the perimeter. The sealed piece remains so until the person peels off the cover 4 at the time of application. As a further alternative construction, the applicator includes non-woven polymer that receives the formulation of art 2 as tattoos by printing.

As a further alternative construction, the applicator includes non-woven polymer that receives the formulation of art 2 as tattoos by printing. In the operations of this disclosure, the textured coating has the cosmetic sample locating within its interstices. Then mutually parallel barrier coatings layer upon and confront the textured coating. The sample remains with the textured coating because of stilting and its repose while the textured coating becomes effectively sealed by the adjacent barrier coatings. This layered arrangement of textured coating and barrier coating does not require a perimeter seal by heat or other welding methods.

The device 1 begins with the components of a body art composition selected by the manufacturer. The composition is then rendered into a state for placement upon a sampler, or piece, as in FIG. 4. The base support 3 can be a printable paper page, sheet of material, or a substrate that may have a generally rectangular shape where the longitudinal axis is longer than the lateral axis. In this figure, the longitudinal axis is oriented upright. The substrate has a fold line, as at 5, centered to allow for convenient gripping by the user when the base support is folded. The base support 3 with the fold line 5 still allows for placement of the cover 4a over the art 2 made of the composition. In an alternate embodiment, the base support 3 has at least one ultraviolet light cured, cationic barrier-type coated surfaces, as at 13 on the left and as at 14 on the right. The coated surface 13, or section of barrier coating, has a substantially smooth surface. In contrast, the opposite coated surface 14 includes a textured surface of known geometry applied upon a barrier coating, as later shown in FIGS. 5-8, and an application of body art composition material 15 within the perimeter of the textured surface. Though a sample material is described broadly, the sample includes fragrance embedded compositions, substantially gelled compositions, and the like, with chemically altered viscosity and surface tension. The compositions include various additives that manipulate the viscosity and surface tension of the composition fragrance solution without affecting its scent.

The body art composition may undergo modification of its viscosity in various ways. Such modifications utilize oils or other fluids to change the resulting viscosity of the composition. Typically, fragrance oil has a viscosity range of about 2 to about 12 centipoise. However, the type of applicator or dispensing equipment may require thickening of the liquid, that is, a higher viscosity, for proper passage through the equipment. Most equipment operates upon compositions having a viscosity between 40 centipoise and 2400 centipoise, however, viscosity in the range of 200,000 centipoise is still accommodated. The liquid fragrance of modified viscosity includes a blend of materials, or the addition of rheology modifiers, emulsions, suspensions, reacted materials, and other forms of thickened liquids. The liquid fragrance of modified viscosity may or may not have adhesive qualities.

The Applicants foresee modifying the composition's viscosity using various components. Those components include blends of cellulose gums, cellulose derivatives, carboxymethylcellulose, sodium carboxymethylcellulose, hydroxypropylcellulose, hydroxyethylcellulose, methylcellulose, ethylcellulose or ethycell; vegetable gums, xanthan gum, acacia gum; alginates, carrageenan, alcogum; silicones, versagels, silicone fluid 200; clays, veegum, bentone gel, silicas, untreated fumed silica or Cabosil® M-5 from Eager Plastics of Chicago, Ill., specially treated fumed silica or Cabosil®TS-720, TS-630; surfactants, sodium lauryl sulfate, ammonium lauryl sulfate; fillers, calcium polycarbophil; emulsions, polyvinyl alcohol or Celvol® from Celanese Corp. of Dallas, Tex.; and suspensions, acrylic acid derivatives such as Carbopol® 940 and Ultrez® 10 from Lubrizol Corp. of Wickliffe, Ohio. One example adjusts the viscosity of the composition by adding ethycell at the rate of 5% by weight and mixing the composition at room temperature under high shear for five hours, which produces a composition with viscosity in the range of 1700 to 1900 centipoise.

In a further alternate embodiment, the body art composition includes a component to minimize the adverse effects of exposure to sunlight, or a sunblock. The sunblocking component within the coating prevents sunlight from reaching the user's skin beneath the component. The device locates the sunblocking component in coordination with the body art, or image, of a tattoo. The sunblocking component within the body art composition creates a reverse image upon the skin of a user that blends non-tanned skin with the body art of the tattoo. The sunblocking component includes titanium dioxide.

Generally, the textured coating section 2 has a pattern of spaced apart cells or a plurality of pockets. The barrier coating or base coat begins with an existing low odor, ultraviolet curable, cationic type varnish. Such a varnish includes RAD-KOTE product number K261 from Actega Radcure of Wayne, New Jersey. This varnish has a viscosity of approximately 375 centipoise. The low odor attribute of this varnish makes it preferable over coatings from other manufacturers. The barrier coating is applied on to a printed web of material using a flexographic coater with a CYREL type printing plate. The printing plate has a smooth finish and is sized to meet the dimension of the desired application. Generally, the barrier coating is applied to the web of material in a thickness of about 0.3 mils to about 0.6 mils, depending on the surface finish or porosity of the web of material, commonly paper or substrate. An about 0.3 mils to about 0.4 mils thick application of base coat is effective on a high quality, smooth finish paper which is used in commercial printing. The coating then undergoes curing at an ultraviolet light curing station mounted directly after the flexographic coater. The intensity of ultraviolet light used relates to the desired operation speed of the press. Generally, printers provide approximately 100 watts of ultraviolet light per every 100 feet per minute of press web speed. As an example, a press running at 1000 feet per minute calls for 1000 watts of ultraviolet light curing capability.

Then an enlarged depiction of the textured coating 14 appears in FIGS. 5-8. FIG. 5 depicts a detailed view of a tight grid, or cross hatch, texture pattern upon the coated surface 14. This pattern has lines intersecting at right angles with the lines of thinner width than the squares of base support between adjacent lines. This pattern provides a suitable application surface for the body art composition, as at 15, along the thin lines between the squares of substrate material.

The texture coating is preferably a low odor, ultraviolet curable, cationic type adhesive. Such an adhesive includes RAD-KOTE product number K6004B from Actega Radcure of Wayne, N.J. This adhesive has a viscosity of approximately 825 centipoise. The Applicants prefer this adhesive for its ability to build height to the texture, as it possesses a greater viscosity and solids content than what is used for the base coat. Though described as an adhesive, the present device has the adhesive cured immediately in a pattern as later shown that builds the texture of the device.

The texture coating is also applied to the material, paper, or substrate, using a flexographic coater with a CYREL type printing plate followed by immediate curing at an ultraviolet station as previously described. This printing plate though has a raised or negative image, of the desired texture pattern in the appropriate size for the desired product. Generally, the texture coating is applied in a thickness ranging from about 0.25 mils to about 2.5 mils depending on the amount of fragrance loaded into the present device. The Applicants prefer a thickness in the range of about 0.5 mils to about 1.25 mils. As an example of single sided texture delivery device includes a one square inch fragrance fluid application upon a 30 line per inch grid texture where the grid has a 1.0 mil height. This example yields a payload of approximately 0.27 fluid drams or about 0.1 milliliter. The device also includes textured coating upon both surfaces which doubles the fragrance payload.

Alternatively, the body art composition is applied by a flexographic coater as previously described. This printing plate though is made of a soft, closed cell foam material, such as Poron®. These plates, or pads, possess a smooth surface and a low memory attribute that enhances application repeatability, usually for adhesive application.

FIG. 6 illustrates a detailed view of an alternate embodiment of the texture pattern as a quad cell-type pattern also upon the coated surface 14. This pattern has individual cells, of substrate material, with rounded corners where the cells are oriented at a forty five degree angle to the edges of the product sampler. The application of liquid fragrance material, as at 15, generally occupies the diamond like shapes between the cells in this figure.

FIG. 7 shows a detailed view of a dot matrix-type texture pattern upon the coated surface 14. Similar to FIG. 2, this pattern also has lines at right angle intersections with the lines having similar width to the squares of substrate between adjacent lines. This pattern has a suitable application surface. for body art composition 15 along the wider lines between the squares of substrate material.

FIG. 8 provides another detailed view but of a random dot pattern of the base coating layer applied to the substrate as the coated surface 14 through the use of an atomizing device. Alternatively, the random dot pattern arises upon mixing a fine aggregate particle material, such as nylon spheres of a certain diameter, into the barrier coating material and applying the mixture upon the substrate to create texture that secures an application of body art composition, as at 15. In a further alternate embodiment, a textured barrier film applied to the cover forms the coated surface 14. In another alternate embodiment, mechanically altered, or distressed, coating film applied to the cover makes the textured coating section. The textured coating section may also have porosity that defines a pattern of texture for retaining liquid fragrance material.

Following the description of the various patterns upon the coated surface 14, FIG. 9 shows the interaction of body art composition with the surface texture in a pattern similar to that shown in FIG. 8. This view is highly magnified, generally showing individual droplets of fragrance secured within the texture, particularly its surface features. The base support 3, often paper, provides a textured mounting surface, as at 14, to which is applied the body art composition, as at 15, here shown between individual cells of texture, as at 14. Opposite the mounting surface or texture 14, the device has its cover 4a. The features of the texture contact the cover and seal the gaps between individual textures. The individual textures modify the behavior of the deposited body art composition, such as at 15 between two adjacent textures 14, so as to defeat capillary action and wicking of any oils from the composition into the base support 3. The textured surface thus occludes the migration, or flow, of the body art composition from its application location through the smooth and the textured surfaces as at 13, 14 and then out of the product sampler. The device achieves stilting between the cover and the mounting surface. In an embodiment with two separate films as the cover and base support, the separate films with the appropriate surface coatings and textures avoid or retard the capillary infiltration of the body art composition into the fibers of the sampler. Further, because the textured surface contains the body art composition, the inability of the fragrance to flow along with its inherent surface tension causes the fragrance material to substantially repose and remain within its locations inside the texture of the barrier coating supplied upon the textured surface 14. Thus, the base support and cover create an occlusive, cohesive seal between the surfaces at each location where body art composition is applied thus removing the need for any perimeter seal.

In the preferred embodiment, the specific ingredients for the formulation for the transfer device of this device include the following:

| Ingredient, INCI/ | | | |
|---|---|---|---|
| Chemical Name | CAS Number | Classification | Ranges |
| Ethylhexyl Palmitate | 29806-73-3 | Oil | 20.0%-45.0% |
| Petrolatum | 8009-03-8. | Oil | 8.0%-12.0% |
| Octyldodecanol | 5333-42-6 | Oil | 3.0%-6.0% |
| Ozokerite | 8021-55-4 | Wax | 5.0%-8.0% |
| Microcrystalline Wax | 63231-60-7 | Wax | 5.0%-10.0% |
| Euphorbia Cerifera | 8006-44-8 | Wax | 1.0%-3.0% |
| (candellilla) Wax | | | |
| VP/Eicosene Copoly- | 28211-18-9 | Emollient | 3.5%-6.0% |
| mer | | | |
| Bis-Diglyceryl | 82249-33-0 | Emollient | 2.0%-4.0% |
| Polyacyladipate - 2 | | | |
| Silica | 7631-86-9 | Feel Enhancer | 2.0%-4.0% |
| Propylparaben | 94-13-3 | Preservative | 0.2%-0.4% |
| Tocopheryl Acetate | 7695-91-2 | Antioxidant | 0.1%-0.2% |
| BHT | 128-37-0 | Antioxidant | 0.05%-0.1% |
| Fragrance | | Fragrance | 0%-25.0% |

This particular product includes a fragranced or unfragranced base, which was developed to be utilized in conjunction with the embossed bump carrier technology developed by the Company, so that the end user may execute a clean transfer of the cosmetic from the applicator to the skin. This is accomplished by applying direct pressure on the applicator to the skin. No. other activation is required. This presents specifics challenges to the formulator. The base is of anhydrous nature in the family of a crystalline solid/stick. It is comprised of oils, emollients, waxes, pigments, shimmers and possibly fragrances. A major step in the production process is to print the cosmetic in its amorphous form. This is accomplished by shearing the product to break its crystalline structure; the product is then rendered printable. A major challenge to this process is due to the cosmetic returning to its crystalline form, whether by reaching its softening point by inadvertent heating and cooling or by the passage of time. This is a crystalline product natural state. As a result, the product no longer becomes a clean transfer and becomes affixes to the applicator. Reformulating by increasing the oils alone does not accomplish the task. The cosmetic loses structure and becomes too runny to support sharp printing and exhibits poor stability. With development, the functional product was achieved by increasing the level of the microcrystalline wax. The normal range of use for this product is 0.5-1.5%. The range at which the formula is formulated is 5-10%. Microcrystalline wax exhibits properties of elasticity, which at high levels turns a product semi crystalline or thixotropic, a result the formulator would consider unstable, but it is this controlled instability that we require to keep the formulation in it amorphous state, which allows the product to function properly. The specific amount of microcrystalline wax required is dependent on the colorants and or fragrances used and thus needs to be adjusted accordingly.

From the aforementioned description, a body art transfer device has been described. The body art transfer device is uniquely capable of retaining a formulation upon a substrate beneath a cover and then depositing the formulation in artistic forms upon the skin of a person. The body art transfer device may be manufactured from many materials, including but not limited to, paper, cardstock, paperboard, polymers, polyethylene terephthalate, ferrous and non-ferrous metal foils and their alloys, and composites.

I claim:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A body art transfer device comprising:
a base support having a generally planar shape, a substantially nonabsorbent textured surface, and a surface area;
a body art coating positioned on the nonabsorbent textured surface, the coating having a pigmented composition of oils and waxes, and a fragrance, having a thickness of uniform homogeneous consistency and an amorphous non-crystalline structure;
a cover for covering the body art coating and for forming stilting of the body art coating between the nonabsorbent textured surface of the base support and the cover, the stilting for preventing wicking of the body art coating, and the body art coating for releasing from the base support when the cover is removed and the body art covering is placed against skin of a user;
the thickness of the body art coating is between about 0.5 mil and 5 mils;
the nonabsorbent textured surface has an irregular texture for receiving the body art coating, said textured surface has a plurality of raised projections which extend through the body art coating;
said base support has a further coating comprising an ultraviolet light cured cationic-type coated surface;
the body art coating has a formulation within the ranges comprising:

| Ingredient, INCI/Chemical Name | Classification | Ranges |
|---|---|---|
| Ethylhexyl Palmitate | Oil | 20.0%-45.0% |
| Petrolatum | Oil | 8.0%-12.0% |
| Octyldodecanol | Oil | 3.0%-6.0% |
| Ozokerite | Wax | 5.0%-8.0% |
| Microcrystalline Wax | Wax | 5.0%-10.0% |
| Euphorbia Cerifera (candellilla) Wax | Wax | 1.0%-3.0% |
| VP/Eicosene Copolymer | Emollient | 3.5%-6.0% |
| Bis-Diglyceryl Polyacyladipate - 2 | Emollient | 2.0%-4.0% |
| Silica | Feel Enhancer | 2.0%-4.0% |
| Propylparaben | Preservative | 0.2%-0.4% |
| Tocopheryl Acetate | Antioxidant | 0.1%-0.2% |
| BHT | Antioxidant | 0.05%-0.1% |
| Fragrance | Fragrance | 0%-25.0% |

thereby providing a transfer of body art that may be applied to the skin of the user during application.

22. The body art transfer device of claim 21, wherein said body art composition being mechanically blended below its melting temperature forming its uniform homogeneous consistency and amorphous structure.

23. A body art transfer device comprising:
a base support having a generally planar shape, a substantially nonabsorbent textured surface, and a surface area;
a body art coating positioned on the nonabsorbent textured surface, the coating having a pigmented composition of oils and waxes, and a fragrance, having a thickness of uniform homogeneous consistency and an amorphous non-crystalline structure;
a cover for covering the body art coating and for forming stilting of the body art coating between the nonabsorbent textured surface of the base support and the cover, the stilting for preventing wicking of the body art coating, and the body art coating for releasing from the base support when the cover is removed and the body art covering is placed against skin of a user;
the thickness of the body art coating is between about 0.5 mil and 5 mils;
the nonabsorbent textured surface has an irregular texture for receiving the body art coating, said textured surface has a plurality of raised projections which extend through the body art coating;
said base support has a further coating comprising an ultraviolet light cured cationic-type coated surface;
the body art coating has a formulation within the ranges comprising:

| Ingredient, INCI/Chemical Name | Classification | Ranges |
|---|---|---|
| Ethylhexyl Palmitate | Oil | 20.0%-45.0% |
| Petrolatum | Oil | 8.0%-12.0% |
| Octyldodecanol | Oil | 3.0%-6.0% |
| Ozokerite | Wax | 5.0%-8.0% |
| Microcrystalline Wax | Wax | 5.0%-10.0% |
| Euphorbia Cerifera (candellilla) Wax | Wax | 1.0%-3.0% |
| VP/Eicosene Copolymer | Emollient | 3.5%-6.0% |
| Bis-Diglyceryl Polyacyladipate - 2 | Emollient | 2.0%-4.0% |
| Silica | Feel Enhancer | 2.0%-4.0% |
| Propylparaben | Preservative | 0.2%-0.4% |
| Tocopheryl Acetate | Antioxidant | 0.1%-0.2% |
| BHT | Antioxidant | 0.05%-0.1% |
| Fragrance | Fragrance | 0%-25.0% |

thereby providing a transfer of body art that may be applied to the skin of the user during application;
wherein the raised projections occupy at least three percent of the surface area of the base support;
the body art coating is screen printed upon the base support, and the screen printing on the base support is applied through a screen having a mesh count of between about 80 to about 420 per linear inch;
wherein the fragrance scented body art composition is formed by mechanical blending below a melting temperature of the scenting body art composition to form the uniform homogeneous consistency and amorphous structure; and
thereby providing a transfer of fragrant body art that may be applied to the skin of the user during application.

24. The body art transfer device of claim 23 wherein the non absorbent textured surface has that irregular texture for receiving the body art composition.

25. The body art transfer device of claim 24 wherein the textured surface has a plurality of raised projections which extends through the body art composition.
